(19) United States
(12) Patent Application Publication
(10) Pub. No.: US 2015/0315618 A1
(43) Pub. Date: Nov. 5, 2015

(54) Enzymatic Chemical Processing in a Fiber Conduit Apparatus

(71) Applicant: John Lee Massingill, San Marcos, TX (US)

(72) Inventor: John Lee Massingill, San Marcos, TX (US)

(21) Appl. No.: 14/744,839

(22) Filed: Jun. 19, 2015

Related U.S. Application Data
(63) Continuation of application No. PCT/US2013/076646, filed on Dec. 19, 2013.

(51) Int. Cl. C12P 7/64

(52) U.S. Cl. CPC C12P 7/649

(57) ABSTRACT

A method is provided which introduces a first stream proximate a plurality of fibers positioned within a fiber conduit apparatus such that the first stream constitutes a phase substantially constrained to the surface of the fibers. The method further introduces an enzyme into the apparatus and introduces a second stream into the apparatus that is in contact with and is substantially immiscible with the first stream. The first stream, the enzyme and the second stream are introduced into the apparatus such that the enzyme interacts with a species from one of the first and second streams and compositions of the first and second streams are altered. An apparatus is provided which includes a plurality of fibers positioned longitudinally within a conduit between two fluid inlets and a fluid outlet, wherein the plurality of fibers have one or more enzymes immobilized thereon.

### PRIORITY CLAIM

This application is a continuation of pending International Application No. PCT/US2013/076646 filed Dec. 19, 2013, which designates the United States and claims priority to U.S. Application No. 61/739,481 filed Dec. 19, 2012.

### BACKGROUND OF THE INVENTION

1. Field of the Invention

This invention generally relates to fiber conduit apparatuses, and specifically relates to enzymatic chemical processing in such devices.

2. Description of the Related Art

The following descriptions and examples are not admitted to be prior art by virtue of their inclusion within this section.

Fiber conduit apparatuses are utilized for a variety of chemical processes in which two essentially immiscible fluids are brought into contact with each other and resulting phases are separated from each other. Advantages of using a fiber conduit apparatus relative to other types of chemical processing include but are not limited to:
(1) Processes are very fast because of very high surface area for mass transfer.
(2) Faster processing allows relatively small fiber conduit apparatuses to be used instead of large open settling zones and/or tanks. As a result, the footprint of the process, the cost and size of the process equipment, and loss of volatile organic compounds will be less, with significant implications for solvent recovery rates and plant safety.
(3) By-products are greatly reduced because dispersions and rag layers (crud) are generally reduced. Since dispersions are reduced, settling time for coalescence of the dispersed particles is reduced, thus reducing collection and processing time and costs, which give way to an even smaller plant foot print.
(4) The fiber conduit apparatus is an extremely effective microchannel extractor/reactor/contactor with the additional benefit of being easily scaled up to any desired volume by simply using larger diameter conduits with more fibers. This is in stark contrast to other traditional “scale-up” approaches, where larger volumes can impact the physical processes and efficiencies involved.

Despite the aforementioned advantages, improvements are needed for some chemical processes to be used in fiber conduit apparatuses. In particular, some chemical processes present difficulties which do not make them cost effective and/or plausible for use in a fiber conduit reactor. In addition, improvements are needed to increase yield, reduce generation of undesirable by-products and/or increase efficiency for chemical processes which have proven viable for use in a fiber conduit apparatus. Accordingly, it would be desirable to develop new chemical processes and components for fiber conduit apparatuses.

### SUMMARY OF THE INVENTION

Processes and apparatuses are provided which employ enzymes in fiber conduit apparatuses. The following description of various embodiments of methods and apparatuses is not to be construed in any way as limiting the subject matter of the appended claims.

Embodiments of methods of chemical processing include introducing a first stream proximate a plurality of fibers positioned within a fiber conduit apparatus, wherein the first stream constitutes a phase substantially constrained to the surface of the fibers. In addition, the methods include introducing an enzyme into the fiber conduit apparatus and introducing a second stream into the fiber conduit apparatus, wherein the second stream constitutes a substantially continuous phase that is in contact with and is substantially immiscible with the first stream. The first stream, the enzyme and the second stream are introduced into the fiber conduit apparatus such that the enzyme interacts with a species from one of the first and second streams and compositions of the first and second streams are altered. The methods further include receiving the altered first and second streams in one or more collection vessels and withdrawing separately the altered first and second streams from the collection vessels.

Embodiments of apparatuses include a conduit comprising at least two fluid inlets and one fluid outlet and a plurality of fibers positioned longitudinally within the conduit between the two fluid inlets and the fluid outlet, wherein the plurality of fibers have one or more enzymes immobilized thereon. The apparatuses further include a collection vessel positioned proximate the fluid outlet.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 illustrates an example of a fiber conduit apparatus useful for the processes described herein;

FIG. 2 illustrates an example of another fiber conduit apparatus useful for the processes described herein;

FIG. 3 depicts an example of a fiber conduit apparatus system useful for the processes described herein; and

FIG. 4 depicts a shell and tube heat exchanger for incorporation into a fiber conduit apparatus.

While the invention is susceptible to various modifications and alternative forms, specific embodiments thereof are shown by way of example in the drawings and will herein be described in detail. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF THE INVENTION

The disclosure herein relates generally to fiber conduit apparatuses, and specifically to enzymatic chemical processing in such devices. More specifically, the disclosure herein is directed to chemical processing using one or more free and/or immobilized enzyme(s) in a fiber conduit apparatus. Although enzymes have been previously employed in many tank and reactor bed chemical processes, enzyme denaturation and survival has been an obstacle, particularly effecting yield and efficiency. For such reasons as well as in view of the intricacies of fiber conduit apparatus processing, it was not expected that enzymes would be effective long enough in a fiber conduit apparatus to sustain a chemical process therein, much less that they would be a viable option (i.e., cost effective, efficient and high yielding) for use in a fiber conduit apparatus. In particular, it was expected that enzymes would die or at least become denaturized quickly in a fiber conduit apparatus given the relatively large interfacial contact area induced by its structure and routing of fluid relative to the fibers (i.e., relative to the interfacial contact area induced in tanks and reactor beds). More specifically, it was not expected that enzymes could survive the fast rate of chemical processing induced in a fiber conduit apparatus (i.e., relative to the rate of processing in tanks and reactor beds). In addition, it was expected that enzymes would be quickly deactivated upon exposure to denaturing chemicals and/or environmental conditions that are involved with many chemical processes. Furthermore, it was not expected that enzymes could survive the pressure levels incurred in a fiber conduit apparatus.

To the contrary, it was discovered during the development of the chemical processes disclosed herein that not only can enzymes and some chemical processes be jointly fashioned to sustain the catalytic activity and/or reactivity of enzymes in a fiber conduit apparatus, the enzymes may be reused for multiple runs through a fiber conduit apparatus without becoming deactivated. As set forth below, the phrase “jointly fashioned” may refer to the selection of the enzyme as well as the selection of the chemical components to be used in the streams. In addition, the phrase “jointly fashioned” may refer to the rate and/or manner at which the enzyme and chemical components are introduced into a fiber conduit apparatus for conducting a chemical process. For example, it was discovered that enzymes may serve as a chemical substitute in some chemical processes and, as a result, the use of harsh chemicals (such as alcohols, strong acids or strong bases) in some chemical processes may be reduced or eliminated, in turn reducing potential sources for denaturing the enzyme. As used herein, a strong acid refers to a composition having a pH of 2.0 or less and a strong base refers to a composition having a pH of 13.0 or greater. Furthermore, it was discovered that the relatively large interfacial exposure and the overall faster processing time incurred in a fiber conduit reactor did not have adverse effects on the catalytic activity and/or reactivity of enzymes. As a result, the flow rates of the fluids introduced into a fiber conduit reactor could be set to conduct an enzymatic chemical process faster than conducting the same enzymatic chemical process in a tank or reactor bed.

Moreover, it was discovered that some enzymes and some chemical processes may be jointly fashioned to advantageously minimize and/or eliminate the formation of a gelatinous emulsion of chemical phases (often organic and aqueous phases) known as crud, gunk, grungies, grumos, or a rag layer which is often an undesirable byproduct of many chemical processes involving contact between two immiscible streams. In particular, some enzymes and some chemical processes may be jointly fashioned to make the contact time needed between immiscible liquids to affect the chemical process be less than the characteristic diffusion time of particles in the liquids and, in turn, the crud problem in a fiber conduit apparatus may be avoided for such a process. Alternatively, an enzymatic chemical process in a fiber conduit apparatus may be fashioned to make the contact time between immiscible liquids to affect the chemical process to be substantially equal or slightly greater than the diffusion time of particles in the liquids and, in turn, the formation of crud may be controlled to a relatively small amount for such a process, particularly an amount which does not cause pressure in the apparatus to exceed a set threshold. As used herein, the phrase “substantially equal” refers to values that are equivalent or which differ by 5% or less. The phrase “slightly greater than” refers to values that are greater than a referenced value by more than 5% and less than 10%.

In general, crud is formed by the diffusion of particles, particularly nanoparticles, from the immiscible liquids into their liquid-liquid interface. In bulk tank chemical processing, the turbulent mixing of the two phases promotes transport of particles to the liquid-liquid interface and, thus, the formation of crud is prevalent. In contrast, free phase flow in fiber conduit apparatuses is laminar and, as a result, transport of particles to a liquid-liquid interface is advantageously much slower. However, for some chemical processes previously conducted in fiber conduit apparatuses, the contact time needed to insure sufficient chemical processing is conducted is longer than the diffusion rate of the particles in the fluids. Thus, crud is produced for some conventional chemical processes conducted in a fiber conduit apparatus. The addition of an enzyme to such chemical processes, however, has shown to reduce and/or eliminate the formation of crud.

Examples of chemical processes which may conducted in the presence of an enzyme in a fiber conduit reactor include but are not limited to covalent chemical transformations, such as, transesterification or esterification of oil and fat triglycerides or fatty acids with short-chain alcohols, to obtain fatty acid short-chain alkyl esters, preferably to be used as biodiesel. Other covalent chemical processes may be applicable as well such as O-alkylation (etherification), N-alkylation, C-alkylation, chiral alkylation, S-alkylation, esterification, transesterification, displacement (e.g., with cyanide, hydroxide, fluoride, thiocyanate, cyanate, iodide, sulfide, sulfite, azide nitrite, or nitrate), other nucleophilic aliphatic or aromatic substitutions, oxidation, hydrolysis, epoxidation & chiral epoxidation, Michael addition, aldol condensation, Cannizzaro reaction, Henry reaction, Wittig condensation, Darzens Condensation, carbene reactions, thiophosphorylation, reduction, carbonylation, transition metal co-catalysis, Mannich reaction, Petasis reaction, Interrupted Feist-Benary reaction, N-heterocyclic carbomethoxylation, hydrogen-transfer reduction, decontamination reactions, HCl/HBr/HOCl/H₂SO₄ reactions, and polymer synthesis or polymer modification. Yet other enzymatic catalyst reactions which may be considered for processing in a fiber conduit apparatus may be reactions used the formation of pharmaceutical products. For example, enzymatic reactions may be processed in a fiber conduit reactor to produce chiral products with high enantiometric purity.

Depending upon the reaction to be conducted, any type and concentration of enzyme may be considered for chemical processing in a fiber conduit apparatus. In particular, any enzyme categorized as an oxidoreductase, transferase, hydrolase, lyase, isomerase, or ligase may be considered. In addition, the enzymes may be isolated enzymes or whole cells. Furthermore, any number of different enzymes may be employed in a single process through a fiber conduit apparatus. Examples of chemical processes are set forth below which employ lipases, particularly phospholipases. More specifically, the use of different phospholipases within a conduit fiber apparatus is described for degumming oil and for the synthesis of fatty acid short-chain alkyl esters (otherwise known as biodiesel) from the transesterification of oil and alcohol. It is contemplated that other lipases may be used for such applications and/or lipases may be used for other applications. For example, triacylglycerol hydrolases (i.e., a subclass of lipases, categorized as E.C. 3.1.1.3) are defined as hydrolytic enzymes that act on the ester linkage in triacylglycerol in aqueous systems to yield free fatty acids, partial glycerides and glycerol. This group of enzymes under low water activity is capable of catalyzing their reverse hydrolysis reaction. This reverse catalytic activity of triacylglycerol hydrolases as well as of other lipases has been widely exploited for the synthesis of valuable compounds that contain ester and amide linkages or other related chemicals containing functional groups such as hydroxyl, carboxylic and amino groups. In particular, lipases have been utilized for reforming fats, oils, waxes, phospholipids and sphingolipids to obtain new desired functional properties, and for separating optically active compounds from their racemic mixtures.

Currently, there are more than 40 different lipases and phospholipases commercially available. A few of them are prepared in commercial quantities. Some of the commercially available enzymes are derived from Bacillus amyloliquefaciens, Aspergillus oryzae, Candida antarctica, Candida rugosa, Rhizomucor miehei, Pseudomonas sp., Rhizopus niveus, Mucor javanicus, Rhizopus oryzae, Aspergillus niger, Penicillium camembertii, Alcaligenes sp., Burkholderia sp., Thermomyces lanuginosa, Chromobacterium viscosum, papaya seeds, and pancreatin. Although the disclosure provided herein emphasizes chemical processing with lipases and particularly phospholipases, the scope of the disclosure is not necessarily so limited. In particular, other chemical processes utilizing other types of enzymes may be employed in a fiber conduit apparatus.

In general, the fiber conduit apparatuses used to employ the process described herein may be utilized as reactors, extractors and/or contactors. Embodiments of fiber conduit apparatuses which may be employed for the processes discussed herein are shown and described in U.S. Pat. Nos. 3,754,377; 3,758,404; 3,992,156; 4,491,565; 7,618,544; and 8,128,825, all of which are incorporated herein by reference to the extent not inconsistent herewith. In general, the fiber conduit apparatuses are structured such that two essentially immiscible fluids may be interfaced for processing, including one phase which preferentially wets the fibers of the conduit apparatus (hereinafter referred to as the “constrained phase”) and another phase which is passed between the fibers exterior to the constrained phase (hereinafter referred to as the “continuous phase”).

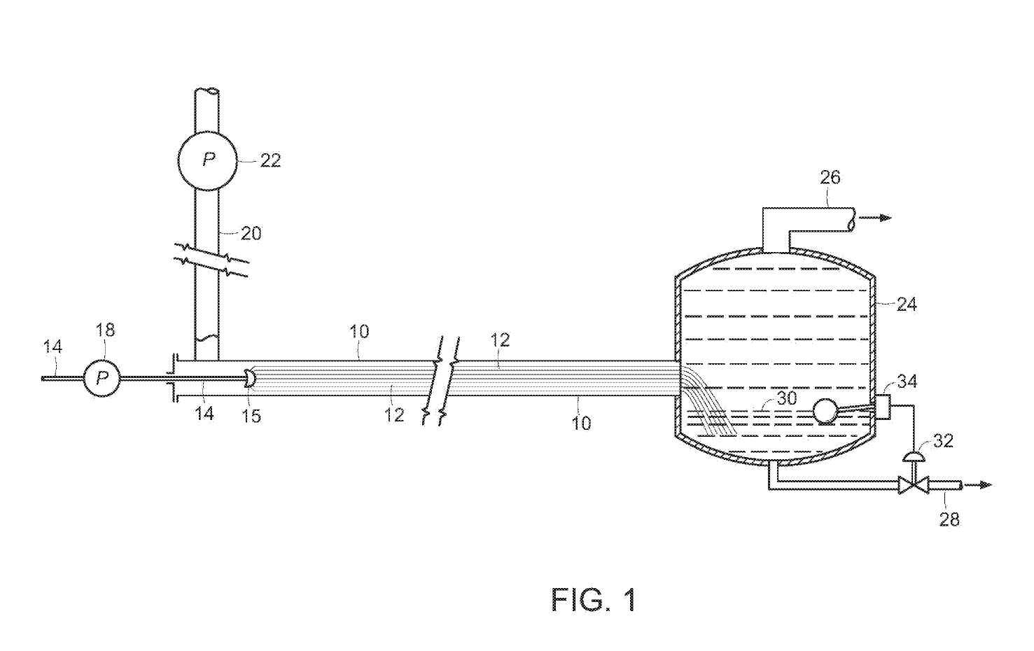

For example, FIG. 1 depicts a fiber conduit apparatus similar to the one disclosed in U.S. Pat. No. 3,977,829—specifically an apparatus having a bundle of elongated fibers 12 filling conduit 10 for a portion of its length. Fibers 12 are secured to tube 14 at node 15. Tube 14 extends beyond one end of conduit 10 and has operatively associated with its metering pump 18 which pumps a first (constrained) phase liquid through tube 14 and onto fibers 12. Operatively connected to conduit 10 upstream of node 15 is inlet pipe 20 having operatively associated with it metering pump 22. This pump 22 supplies a second (continuous) phase liquid through inlet pipe 20 and into conduit 10, where it is squeezed between the constrained coated fibers 12. At the downstream end of conduit 10 is gravity separator or settling tank 24 into which the downstream end of fibers 12 may extend. Operatively associated with an upper portion of gravity separator 24 is outlet line 26 for outlet of one of the liquids, and operatively associated with a lower portion of gravity separator 24 is outlet line 28 for outlet of the other liquid, with the level of interface 30 existing between the two liquids being controlled by valve 32, operatively associated with outlet line 28 and adapted to act in response to a liquid level controller indicated generally by numeral 34.

Although the fiber conduit apparatus shown in FIG. 1 is arranged such that fluid flow traverses in a horizontal manner, the arrangement of the fiber conduit apparatuses considered for the processes described herein are not so limited. In particular, in some cases, the fiber conduit apparatuses considered for the processes described herein may be arranged such that inlet pipes occupy an upper portion of the apparatus and a settling tank occupies the bottom portion of the apparatus. For example, the fiber conduit apparatus shown in FIG. 1 may be rotated approximately 90° in parallel with the plane of the paper to arrange inlet pipes 14 and 20 (as well as node 15) and settling tank 24 in the respective upper and lower positions. Such an arrangement may capitalize on gravity forces to aid in propelling fluid through the apparatus.

In yet other embodiments, the fiber conduit apparatuses considered for the processes described herein may be arranged such that inlet pipes occupy a lower portion of the apparatus and a settling tank occupies an upper portion of the apparatus. For example, the fiber conduit apparatus shown in FIG. 1 may be rotated approximately 90° in the opposite direction parallel with the plane of the paper such that inlet pipes 14 and 20 and node 15 occupy the bottom portion of the apparatus and settling tank 24 occupies the upper portion of the apparatus. In such cases, it was discovered that the hydrophilicity, surface tension, and repulsion of the continuous phase fluid will keep the constrained phase fluid constrained to the fibers regardless of whether the fluids are flowing up, down, or sideways and, thus, sufficient contact can be attained to affect the desired chemical process without the need to counter gravity forces. In yet other embodiments, a fiber conduit apparatus may be arranged in a slanted position for any of the processes described herein (i.e., the sidewalls of the fiber conduit apparatus may be arranged at any angle between 0° and 90° relative to a floor of a room in which the fiber conduit apparatus is arranged).

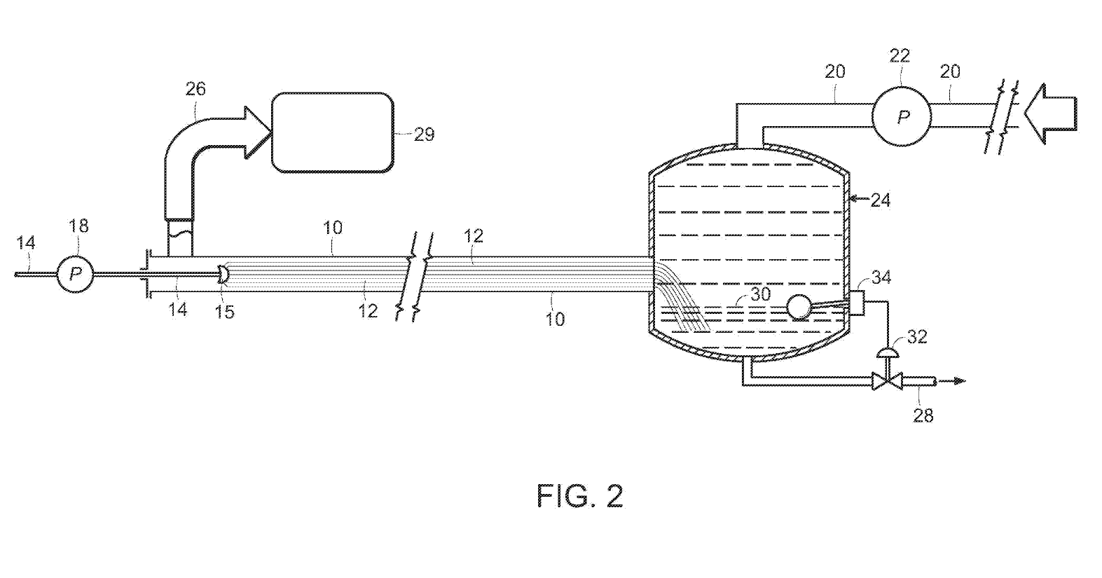

In an alternative embodiment, a counter-current fiber conduit apparatus may be used for the processes described herein. An example of a counter-current fiber conduit apparatus is illustrated in FIG. 2. In particular, FIG. 2 illustrates an alternative configuration of the fiber conduit apparatus depicted in FIG. 1, specifically that the locations of inlet pipe 20 and associated pump 22 have been switched with outlet line 26 to affect counter-current flow of the continuous phase relative to the flow of the constrained phase in conduit 10. In particular, inlet pipe 20 and associated pump 22 in FIG. 2 are connected to an upper portion of settling tank 24 to introduce a second (continuous) phase liquid into settling tank 24 and conduit 10, where it is squeezed between the fibers 12 coated with the first constrained phase liquid. In addition, the fiber conduit apparatus depicted in FIG. 2 includes outlet 26 for the discharge of the second continuous phase liquid from conduit 10 into collection tank 29. An optional addendum to the fiber conduit apparatus configuration depicted in FIG. 2 would be to add an extension line from pipe 20 to conduit 10 near its port to settling tank 24. Such an additional extension line may be used to feed the second (continuous) phase liquid into conduit 10 while bypassing settling tank 24. In any case, due to the configuration of the apparatus depicted in FIG. 2, the size of its settling tank 24 may be optionally reduced by up to 50% relative to the size used for the fiber conduit apparatus depicted in FIG. 1.

Similar to the fiber conduit apparatus depicted in FIG. 1, the fiber conduit apparatus depicted in FIG. 2 includes conduit 10 having a bundle of elongated fibers 12 secured to tube 14 at node 15 and extending a portion of the length of conduit 10. Tube 14 extends beyond one end of conduit 10 and has operatively associated with its metering pump 18 which pumps a first (constrained) phase liquid through tube 14 and onto fibers 12. At the other end of conduit 10 is settling tank 24 into which fibers 12 extend and unload the first phase liquid. Outlet line 28 is arranged at a lower portion of settling tank 24 for discharge of the first phase liquid as controlled by valve 32, which operates in response to level monitor 34 arranged at interface 30. In any case, with the counter-current fiber conduit apparatus configuration depicted in FIG. 2, it was discovered that the hydrophilicity, surface tension, and repulsion of the continuous liquid phase will keep the constrained phase liquid constrained to the fibers even when the constrained phase liquid is flowing in the opposite direction. Such a phenomenon is true in cases in which the constrained phase liquid is flowing up, down, or sideways and, thus, although the counter-current fiber conduit apparatus shown in FIG. 2 is arranged such that fluid flow traverses in a horizontal manner, the arrangement of the fiber conduit apparatus is not so limited. In particular, the counter-current fiber conduit apparatus shown in FIG. 2 may be rotated approximately 90° in either direction parallel with the plane of the paper to respectively arrange inlet pipe 14 and settling tank 24 in upper and lower positions of the apparatus or vice versa. In yet other embodiments, the fiber conduit apparatus depicted in FIG. 2 may be arranged in a slanted position (i.e., the sidewalls of the fiber conduit apparatus may be arranged at any angle between 0° and 90° relative to a floor of a room in which the fiber conduit apparatus is arranged).

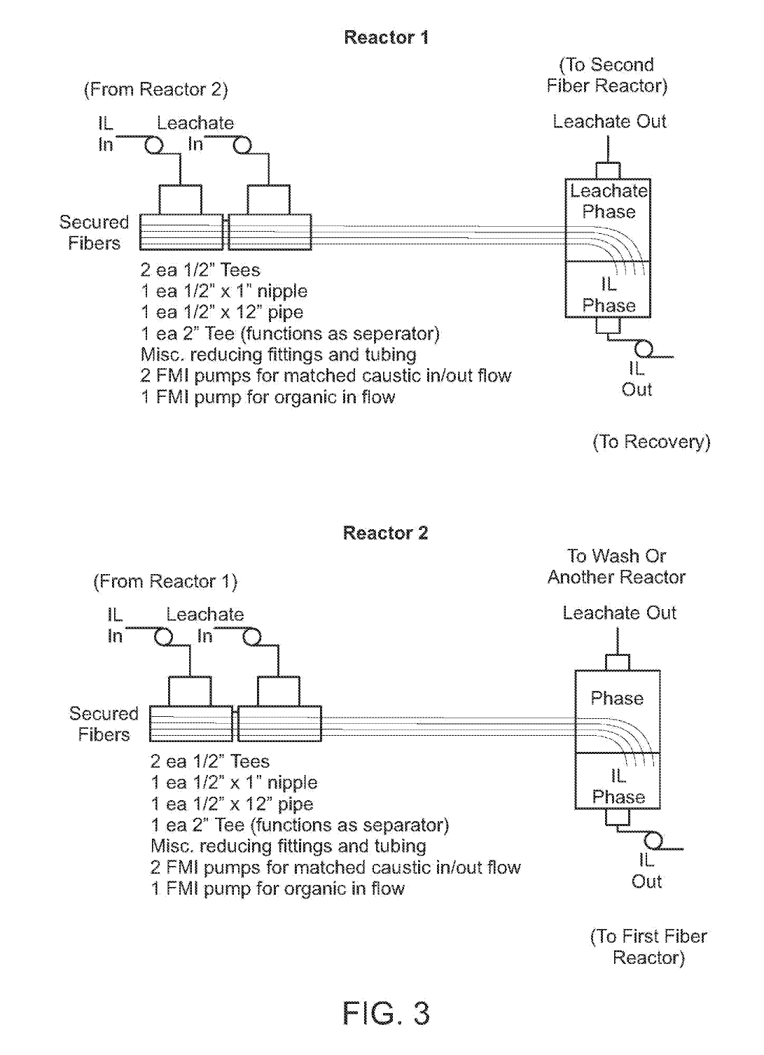

FIG. 3 shows a fiber conduit apparatus system useful for the processes described herein. In operation, the secured fibers in Reactors 1 and 2 are wetted by the constrained phase (denoted in FIG. 3 as “1st stream in”) before the mobile phase (denoted in FIG. 2 as “2nd stream in”) is started. FIG. 3 shows how multiple fiber reactors can be used to increase efficient use of reactants by essentially feeding the liquids counter-currently through the reactor sequence. The continuous phase output of Reactor 1 (denoted in FIG. 3 as “2nd stream out”) is introduced to Reactor 2 (denoted in FIG. 3 as “2nd stream in”) and further processed thereby. The constrained phase output of Reactor 2 is introduced to Reactor 1 (“1st stream in”) while the constrained phase output of Reactor 1 is processed or alternatively introduced to another reactor upstream of Reactor 1 (not shown). In FIG. 3, the constrained and mobile phases are depicted as flowing co-currently through each individual reactor, but the constrained and continuous phases may flow counter-currently through the reactor sequence. In some cases, a fresh composition or a different composition can be used for the first steams introduced into each reactor if desired.

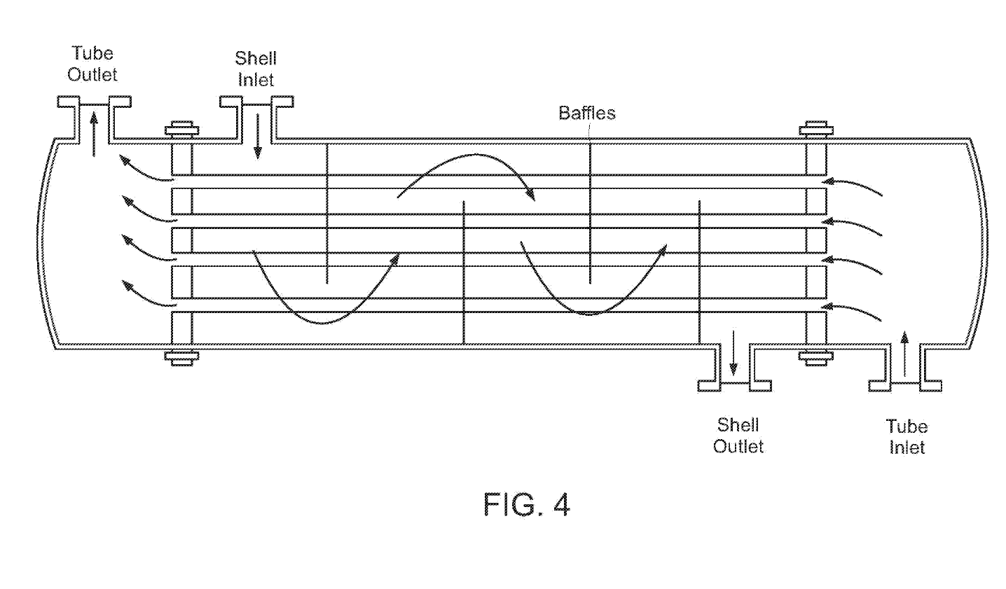

FIG. 4 shows a conventional shell and tube heat exchanger. Combining this design with a fiber conduit apparatus yields a fiber conduit apparatus design (not shown) adapted to handle chemical processes that need to be cooled or heated. One can see that modification of the inlet of the heat exchanger tubes (“Tube Inlet”) to duplicate the inlets shown in FIG. 1 would make each tube in the exchanger act like a thermally controlled fiber reactor (not shown). The exit end of the heat exchanger (“Tube Outlet”) can be modified to operate as a separator (not shown) to collect one phase on the bottom near the end of the fibers (not shown) and allow the other phase to exit from the top of the separator section. Introduction of a heat exchange medium to the exchanger (via “Shell Inlet”) and outflow thereof (via “Shell Outlet”) allows for the addition or removal of thermal energy from the exchanger tubes. While FIG. 4 depicts a counter-current flow heat exchanger, a co-current arrangement could also be used for the processes described herein. In addition, although baffles are shown on the shell side of the exchanger in FIG. 4, the apparatus is not so limited and a heat exchanger without baffles may be employed.

Regardless of the type of fiber conduit apparatus employed for the processes described herein, the fibers may, in some cases, be longitudinal and extend substantially parallel to the sidewalls of the reactor conduit. Other fiber configurations, however, may be considered. In particular, in some embodiments, the fibers may be arranged off angle relative to the conduit sidewalls (i.e., not parallel) (e.g., the fibers may extend from an off-center location at the top of the pipe to the bottom center or to a bottom opposing sidewall or vice versa, etc.). In addition or alternatively, the fibers may be crimped (i.e., zig zag), spiral wound, and/or intertwined (e.g., similar to steel wool cleaning pads stuffed in a pipe). In some embodiments, the fibers may have a circular cross-section, but other cross-sectional shapes may be considered, such as but not limited to elliptical, triangular, square, rectangular, dog-bone, bean-shaped, multi-lobular, and polygonal. In some cases, the fibers may be scaled or serrated. In other embodiments, the exterior surfaces of the fibers may be smooth. In some cases, the fibers can be threads made of relatively short fibers twisted together. In other embodiments, the fibers may be configured similar to a treelike structure with a main fiber and various size limbs and branches attached to the main trunk. Multifilament fibers (textile threads) and less symmetrical monofilaments have greater possibility for dispersions created in the exiting free phase, so it would be preferable to use symmetrical monofilament fibers, but reactions/extractions still occur using multifilament non-symmetrical fibers and any resulting dispersions may be generally manageable in practice. In any case, the configuration of the fibers (e.g., shape, size, number of filaments comprising a fiber, symmetry, asymmetry, etc.) within a fiber conduit apparatus may be the same or different for the processes described herein.

The material of fibers for the processes described herein may be generally compatible with the enzyme employed within the fiber conduit apparatus. Examples of materials include but are not limited to cotton, jute, silk, treated or untreated minerals, metals, metal alloys, treated and untreated carbon allotropes, polymers, polymer blends, polymer composites, nanoparticle reinforced polymer, combinations thereof, and coated fibers thereof for corrosion resistance or chemical activity. In addition to being selected for its compatibility with an enzyme, the fiber type is generally selected to match the desired constrained phase. For example, organophilic fibers may be used with a constrained phase that is substantially organic. This arrangement can, for example, be used to extract organic materials from water with organic liquids constrained to the fibers. Suitable treated or untreated minerals for fiber materials include, but are not limited to, glass, alkali resistant glass, E-CR glass, quartz, asbestos, ceramic, basalt, combinations thereof, and coated fibers thereof for corrosion resistance or chemical activity. Suitable metals include, but are not limited to, iron, steel, stainless steel, nickel, copper, brass, lead, thallium, bismuth, indium, tin, zinc, cobalt, titanium, tungsten, nichrome, zirconium, chromium, vanadium, manganese, molybdenum, cadmium, tantalum, aluminum, anodized aluminum, magnesium, silver, gold, platinum, palladium, iridium, alloys thereof, and coated metals.

Suitable polymers include, but are not limited to, hydrophilic polymers, polar polymers, hydrophilic copolymers, polar copolymers, hydrophobic polymers/copolymers, non-polar polymers/copolymers, and combinations thereof, such as polysaccharides, polypeptides, polyacrylic acid, polyhydroxybutyrate, polymethacrylic acid, functionalized polystyrene (including but not limited to, sulfonated polystyrene and aminated polystyrene), nylon, polybenzimidazole, polyvinylidenedinitrile, polyvinylidene chloride and fluoride, polyphenylene sulfide, polyphenylene sulfone, polyethersulfone, polymelamine, polyvinyl chloride, polyvinylacetate, polyvinylalcohol, co-polyethylene-acrylic acid, polyethylene terephthalate, ethylene-vinyl alcohol copolymers, polyethylene, polychloroethylene, polypropylene, polybutadiene, polystyrene, polyphenol-formaldehyde, polyurea-formaldehyde, polynovolac, polycarbonate, polynorbornene, polyfluoroethylene, polyfluorochloroethylene, polyepoxy, polyepoxyvinylester, polyepoxynovolacvinylester, polyimide, polycyanurates, silicone, liquid crystal polymers, derivatives, composites, nanoparticle reinforced, and the like.

In some cases, enzymes may be immobilized on the fibers for chemical processing within a fiber conduit apparatus. In particular, enzymes may be immobilized on the exterior surface of the fibers prior to the introduction of process streams into the fiber conduit apparatus for a particular chemical process. The immobilization of the enzymes may be conducted by any immobilization technique known in the art, such as but not limited to physical adsorption, adsorption on ion-exchange resins, covalent binding (such as to epoxidized polymers), entrapment in a growing polymer, confinement in a membrane or in semi-permeable gels, or cross-linking enzyme crystals (CLECS's) or aggregates (CLEAS's). In some embodiments, enzymes can be converted to a protein fiber (similar to silk), which would be suitable for use in the fiber reactor. In many cases, activity of immobilized enzymes is longer than enzymes in solution (less denaturing of enzymes when they are immobilized) and, thus, use of enzymes immobilized on fibers may be preferred.

The aforementioned disclosure, however, does not preclude the option of introducing an enzyme solution into a fiber conduit apparatus in addition or alternative to immobilizing enzymes on fibers. In some cases, the enzyme solution may be distinct from the other reactive streams introduced into a fiber conduit apparatus for a chemical process. In other cases, however, enzymes may be added to one or more of such streams prior to being introduced into a fiber conduit apparatus. In yet other embodiments, one or more enzymes may be immobilized within a solution introduced into the fiber conduit apparatus. In particular, a suspension may be introduced into the fiber conduit apparatus having particles with one or more enzymes immobilized thereon. In such cases, it would be advantageous to minimize the size of the particles such that plugging the reactor with the particles can be avoided. In particular, it would be advantageous for the particles to have a diameter smaller than the distance between the fibers. In many cases, the density of fibers in a fiber conduit apparatus is such that the distance between fibers in a fiber conduit reactor is approximately equal to the diameters of the fiber, particularly when the fiber packing occupies about 50% of the volume of the conduit. The spacing between fibers, however, may be larger or smaller in some embodiments. It is noted that although it is advantageous for the particles to have a diameter smaller than the distance between the fibers, it is not a necessity. In particular, the particles may, in some cases, have a diameter up to 15% greater than the distance between the fibers since the fibers hang freely in the conduit and can be pushed fibers sideways by the particles carried in the flowing fluid/s to accommodate the particles.

In any case, fibers can, in some embodiments, be treated for wetting with preferred phases, to protect from corrosion by the process streams, and/or coated with a functional polymer. For instance, carbon fibers can be oxidized to improve wettability in aqueous streams and polymer fibers can display improved wettability in aqueous streams and/or be protected from corrosion by incorporation of sufficient functionality into the polymer, including but not limited to, hydroxyl, amino, acid, base, enzyme, or ether functionalities. In some cases, the fibers may include a chemical bound (i.e., immobilized) thereon to offer such functionalities. In some embodiments, the fibers may be ion exchange resins, including those suitable for hydroxyl, amino, acid, base or ether functionalities. In other cases, glass and other fibers can be coated with acid, base, or ionic liquid functional polymer. As an example, carbon or cotton fibers coated with an acid resistant polymer may be applicable for processing strong acid solutions.

In some embodiments, all the fibers within a fiber conduit apparatus may be of the same material (i.e., have same core material and, if applicable, the same coating). In other cases, the fibers within a fiber conduit apparatus may include different types of materials. For example, a fiber conduit apparatus may include a set of polar fibers and a set of non-polar fibers. Other sets of varying materials for fibers may be considered. As noted above, the configuration of fibers (e.g., shape, size, number of filaments comprising a fiber, symmetry, asymmetry, etc.) within a conduit reactor may be the same or different for the processes described herein. Such variability in configuration may be in addition or alternative to a variation of materials among the fibers. In some embodiments, different types of fibers (i.e., fibers of different configurations and/or materials) may run side by side within an apparatus with each set having their own respective inlet and/or outlet. In other cases, the different types of fibers may extend between the same inlet and outlet. In either embodiment the different types of fibers may be individually dispersed in the conduit apparatus or, alternatively, each of the different fiber types may be arranged together. In any case, the use of different types of fibers may facilitate multiple separations, extractions, and/or reactions to be performed simultaneously in a fiber conduit apparatus from a singular or even a plural of continuous phase streams. For example, in a case in which a conduit apparatus is filled with multiple bundles of respectively different fiber types each connected to its own constrained phase fluid inlet and arranged off-angle, the bundles could be arranged for the continuous phase fluid to pass sequentially over the multiple fiber bundles with different materials extracted by or from each bundle.

In general, the processes described herein introduce two essentially immiscible fluids into a fiber conduit apparatus, with one stream preferentially wetting the fibers in the apparatus (hereinafter referred to as the “constrained phase”) and the other stream passing between the fibers exterior to the constrained phase (hereinafter referred to as the “continuous phase”). Depending on the process employed, an additional catalyst, solvent or cosolvent may be included in either of the streams or introduced into the fiber conduit apparatus separate from the two streams. Useful phase transfer catalysts for reactions include, but are not limited to, quaternary ammonium compounds, quaternary phosphonium compounds, sulfonium compounds, crown ethers, polyglycols, and combinations thereof. In any case, the phases discharged from the fiber conduit apparatus may be separately withdrawn and, in some cases, either or both phases may be further processed in the same fiber conduit apparatus, a different fiber conduit apparatus, or another processing apparatus. Examples of subsequent processing may include but is not limited to washing, separation and/or extraction.

As noted above, enzymes may, in some cases, be immobilized on the fibers prior to introduction of the streams. In addition or alternatively, enzymes may be introduced into the fiber conduit apparatus via a fluidic stream. In some of such cases, an enzyme solution distinct from the other reactive streams may be introduced into the fiber conduit apparatus, i.e., prior to, during, or after introduction of one or both of the reactive streams. In other embodiments, however, enzymes may be added to one or more of reactive streams prior to being introduced into a fiber conduit apparatus. In embodiments in which different enzymes are used in a process, the different enzymes may be introduced into the fiber conduit apparatus in the same manner or in different manners.

An example operation of a fiber conduit apparatus is provided in reference to FIG. 1. In particular, a first stream is introduced through tube 14 and onto fibers 12. Another stream is introduced into conduit 10 through inlet pipe 20 and through void spaces between fibers 12. As described in more detail below, any suitable materials comprising substantially immiscible phases may be employed for the two streams. As noted above, enzymes may be immobilized on the fibers prior to introduction of the streams. In addition or alternatively, enzymes may be introduced into the fiber conduit apparatus via a fluidic stream. In either case, fibers 12 will be wetted by the first stream preferentially to the second stream. In particular, the first stream will form a film on fibers 12, wherein the film will be dragged downstream through conduit 10 by the passage of the other stream therethrough. As a consequence of the relative movement of the other liquid with respect to the film on fibers 12, a new interfacial boundary between the two streams is continuously being formed, thus causing and accelerating the chemical process. A catalyst or co-solvent may be optionally employed to facilitate mass transfer across the interface between the phases.

In any case, both phases will be discharged into separator 24. Because the liquid phases come out of the fiber conduit apparatus separated and the constrained phase follows the fibers, the processes described herein may be utilized when the phases are very close in density or when they have a great variance in density. Although the embodiment shown in FIG. 1 describes an arrangement wherein the downstream end of fibers 12 extends into separator 24 below interface 30 so that the heavier liquid can be collected directly in the bottom of separator 24 without it being dispersed into the other liquid, the arrangement of fibers 12 is not so limited. In particular, in some embodiments, the downstream end of fibers 12 within separator 24 may be alternatively disposed above or at the interface between the liquid phases within separator 24, depending on the relative density of the constrained phase and the continuous phase.

The constrained phase of a process conducted in a fiber conduit apparatus can include any liquid that wets the fibers preferentially to the continuous phase, including but not limited to, such materials as organophosphorus acids, water, water solutions, water and co-solvents, alcohols, phenols, amines (including but not limited to, polyamines, ethanolamines, and polyethanolamines), carboxylic acids, ethers, esters, dimethyl sulfoxide, sulfone, dimethyl formamide, ketones, aldehydes, saturated and unsaturated aliphatic hydrocarbons, aromatic hydrocarbons, silicone containing fluids, halogenated solvents, liquefied gases, sulfuric acid, other mineral acids, liquid metals/alloys, and ionic liquids. The scope of the ionic liquids which may be utilized in the methods described herein is set forth in detail below. The continuous phase of a process conducted in a fiber conduit apparatus can include any liquid immiscible with the selected constrained phase. In some cases, immiscible ionic liquids can be used together, one as a constrained phase and one as a continuous phase.

For extraction processes, the constrained phase frequently comprises the extractant, but functionalities of the constrained phase and the continuous phase can be reversed if desired by reversing the polarity of the fibers chosen for a particular separation. In some cases, a solvent may be the extractant. In other embodiments, an extractant may be mixed with a solvent (i.e., the solvent may be used as a carrier medium for the extractant). In either case, an extractant is frequently diluted in another solvent. Examples of diluted extractants which may be used for some processes include but are not limited to Ionquest-801 (an organophosphorus acid) diluted in an aliphatic organic compound; 1-phenyl-3-methyl-4-benzoly-5-pyrazolone (HPMBP) as the extractant in aqueous-chloroform; D2EHPA, acetylacetone and 1,10-phenanthroline in nonpolar organic solvents. In some embodiments, the phase used for extraction may include two immiscible liquids to affect selective extraction for multiple entities. For instance, a continuous phase of two immiscible liquids may be used to extract different species from a fluid stream in the constrained phase or vice versa. Such a process may be advantageous to avoid having to process (i.e., wash) an extractant solution discharged from a fiber conduit apparatus. In some cases, two immiscible ionic liquids may be used to affect selective extraction of entities.

The term ionic liquid (IL) is used herein to refer to a salt in a liquid state. In some cases, the term is specific to salts having a melting point below 100° C. ILs are also known as liquid electrolytes, eutectic mixtures, ionic melts, ionic fluids, or liquid salts. An advantage of ILs is their high solvation ability for compounds of widely varying polarity. Furthermore, utilizing ILs is one of the goals of green chemistry because ILs potentially create a cleaner and more sustainable chemistry as environmental friendly solvents for many extractive, reactive, and catalytic processes. Moreover, utilizing ILs offer potential improvement in process economics, chemical reactivity, selectivity, and yield. As such, it may be particularly advantageous, in some cases, to employ ionic liquids for the processes described herein. ILs are usually formed by a large organic cation combined with an anion of smaller size and more symmetrical shape, although some symmetric cations are also combined with asymmetric anions to form ionic liquids. In spite of their strong charges, their asymmetry prevents them from solidifying at low temperatures. Furthermore, ionic liquids can be made hydrophilic or hydrophobic. For example, an eutectic mixture of choline chloride (i.e., MCl₂, wherein M=Zn or Sn) forms a moisture stable Lewis acidic ionic liquid.

Some common cations which may be considered for the formation of ILs employed herein are imidazolium, benzotriazolium, pyrrolidinium, piperidinium, pyridinium, isoquinolinium, thiazolium, sulfonium, ammonium, phosphonium and aminium, but other cations may be considered. Some common anions which may be considered for the formation of ILs employed herein are halide, borate, carbon icosahedral, nitrite, amides, imides, nitrate, hydrofluoride anions, aluminate, mesylate, sulfate, sulfinates, sulfonates, tosylate, sulfate, phosphate, acetate, alkanoates, aluminate, arsenic, niobium, tantalum and trisubstituted methane, but other anions may be considered. In particular, a comprehensive database from literature date between 1980 and 2004 has been published denoting 276 kinds of cations and 55 kinds of anions suitable for IL formation (“Physical Properties of Ionic Liquids: Database and Evaluation,” J. Phys. Chem. Ref. Data, Vol. 35, No. 4, 2006).

ILs are advantageous because they can be tuned with a well-judged selection of the cation-anion pair, giving the opportunity to choose among a vast range of different ionic liquids. In particular, hundreds of ionic liquids have been synthesized and there is virtually no limit in the number of possible counter-ion pairs and mixtures of them that can be obtained. In fact, the number of possible ionic liquids is estimated around 10¹⁸, whereas the number of traditional solvents widely used in industry is only a few hundred. ILs based on a specific organic cation and/or anion for several potential specific applications are known, examples of which include chiral ionic liquids (using natural or synthesized chiral units) for asymmetric catalytic transformations, enantioselective resolution or separation processes; pharmaceutical ionic liquids (called API-ILs incorporating an active principle ingredient as cation or anion); magnetic ionic liquids (based on FeCl₄ anions) for efficient separation processes; and as intrinsically functional materials (for example luminescent, photochemical or electrochemical ILs).

In addition, IL compounds can also be tuned by the modification of the cation and/or the anion molecular structure adding appropriate functional groups in order to obtain ionic liquids with a set of desired physico-chemical properties, which are known as task specific ionic liquids (TSIL). In particular, supramolecular structure and organization have emerged as important and complicated topics that may be key to understanding how chemical reactions and other processes are affected by ionic liquids. In general, TSILs may be developed with desired physico-chemical properties such as density, thermal/electrical conductivity, viscosity, polarity, and non-toxic or biodegradable ILs. For instance, it has been reported that replacing one atomic element in an ionic species with another heavier element affects the physical and chemical properties of ILs in unexpected ways. For instance, comparison of ILs with C and Si in a side group of 1-methyl-3-neopentylimidazolium and 1-methyl-3-trimethylsilyl-methyl-imidazolium with the same anion showed that shear viscosities of the silicon substituted ILs were substantially less than those of the respective carbon ILs. Heavy atom substitution also affects the static properties such as liquid density, shear viscosity, and surface tension. This feature of ILs is the opposite of that observed in conventional neutral molecular liquids.

Computer modeling tools are being developed that will enable ILs to be designed for specific tasks. Two different and complementary approaches have shown excellent predictive power: (1) the soft-SAFT equation of state, used to predict the solubility of several compounds in different families of alkylimidazolium ionic liquids, as well as interfacial properties, and (2) classical molecular dynamic simulations, used to study transport properties like self-diffusion, viscosity and electrical conductivity of ionic liquids. These tools help in getting additional insights into the underlying mechanisms governing the behavior of these systems, which is the basic knowledge needed for a rational design of TSILs. It is noted that ILs may be advantageous for any of the applications disclosed herein.

As noted above, the incorporation of enzymes into chemical processes performed in a fiber conduit apparatus may be particularly applicable to processes relating to the production of biodiesel. For instance, the use of enzymes in a fiber conduit apparatus may be desirable for degumming processes in which phosphatides are removed from a vegetable oil or fat. In general, phosphatides can interfere with a transesterification process used to produce biodiesel and, thus, they must be removed prior to such a process. It is noted that degumming vegetable oil or fats is not limited to precursor processes to the production of biodiesel, but is applicable for other applications as well, such as preparing lecithin or preparing refined vegetable oil suitable for edible consumption. It is also desirable to degum oil for long-term storage since wet gums precipitate over time and cause sludge build-up. In any case, the oil/fat stream will generally be introduced into the fiber conduit apparatus as the continuous phase and the extractant stream will be introduced as the constrained phase, but the reverse of such may be conducted if organic fibers are employed in the apparatus.

Conventional degumming operations utilize water/acid or water/caustic followed by settling or centrifugation. When caustics are used, the process simultaneously neutralizes the vegetable oil or fat, i.e., removes free fatty acids from the oil or fat. Such a combo degumming and neutralization process, however, results in an emulsion called soap stock. Multiple centrifuging steps are needed to remove the soap stock and associated costs are significant. Also, soap stock is a low-value by-product. Furthermore, this process undesirably removes potential biodiesel precursor fatty acids. In view of this, water/acid degumming operations are sometimes preferred. In any case, strong acids and bases are typically used in conventional degumming operations in order to effect an efficient reaction. As used herein, a strong acid refers to a composition having a pH of 2.0 or less and a strong base refers to a composition having a pH of 13.0 or greater. Use of strong acids and bases is a safety hazard and the efforts and costs for neutralizing such for disposal is generally higher than weaker acids and bases. Furthermore, when a solvent is used in a conventional degumming operation, such as use of ethanol with an acid, the solvent generally needs to be distilled and recycled after the operation. Moreover, the time required for reaction and subsequent settling and/or centrifugation for conventional degumming operations is generally extensive, on the order of a few to several hours.

Performing degumming operations in a fiber conduit apparatus in the presence of enzymes mitigates much of the disadvantages of conventional degumming operations. In particular, it has been discovered that a process of refining vegetable oils or fats (particularly crude (i.e., unrefined) vegetable oils or fats) by enzymatic dephosphorylation in a fiber conduit apparatus can be performed in a short amount of time (i.e., on the order of a few minutes, depending on the length of the fibers). For example, such a process may be performed in less than 10 minutes and, in some cases, less than 5 minutes in a 0.5 inch outer diameter apparatus containing 12 inch fibers. The flow rates of the oil and the extracting stream may generally range between approximately 0.1 ml/min and approximately 4.0 ml/min and the ratio of the flow rates (i.e., oil stream:extract stream) may generally range between approximately 1:1 and approximately 5:1 and, in some specific cases, may be approximately 2:1. The term “approximately” as used herein refers to variations of up to +/−5% of the stated number. Moreover, in using the aforementioned flow rates and ratios thereof, the pressure of a fiber conduit apparatus did not substantially increase during the process, indicating relatively little or no formation of crud. In general, the pressure of a fiber conduit reactor may be any pressure the conduit and pumps of the apparatus are designed to handle. An example range which may be used is between approximately 30 psi and approximately 500 psi, depending on the size of the conduit and pumps.

In addition, it was discovered that enzymatic dephosphorylation of vegetable oils or fats (particularly crude (i.e., unrefined) vegetable oils or fats) in a fiber conduit apparatus can be performed with no strong acids or strong bases (and, in some cases, void of any acids, bases, or non-aqueous solvents) and without the need for subsequent distillation or centrifugation steps. In particular, it was discovered that streams introduced into a fiber conduit apparatus for enzymatic dephosphorylation of vegetable oils or fats are compositions having pHs between 2.1 and 12.9. In some embodiments, the oil/fat stream may consist essentially of vegetable oil and the extractant stream may be an aqueous stream. Further to such cases, the extractant stream may, in some embodiments, consist essentially of the enzyme and deionized water. In other cases, however, the extractant stream may have the enzyme immobilized on a suspended particle in the solution or, alternatively, the enzyme may be immobilized on the surface of the fibers. The concentration of an enzyme in a solution introduced into a fiber conduit apparatus for the dephosphorylation of vegetable oils or fats may be generally greater than or equal to approximately 0.001 wt % and, in particular cases, may be between approximately 0.001 wt % and approximately 0.200 wt. %. Furthermore, it was discovered that enzymatic dephosphorylation of vegetable oils or fats in a fiber conduit apparatus may be conducted at a relatively moderate temperature between approximately 20° C. and approximately 75° C., more specifically between approximately 45° C. and approximately 60° C.

In general, the process converts phospholipids in vegetable oil or fats to oil soluble diglycerides and water soluble phosphate derivatives. Due to their water solubility, the phosphate derivatives will transfer out of the vegetable oil to the opposing stream along the route of the fibers through the apparatus. As a result, a refined vegetable oil with little or no phosphates is discharged into the collection vessel of the fiber conduit apparatus, making a subsequent settling or centrifugation step unnecessary. More specifically, over two thirds of the gum molecule is retained in the oil by such a process and extracts undesirable phosphate derivates, such as choline phosphate, inositol phosphate, serine phosphate, ethanol amine phosphate, glycerol, and phosphate, into the other fluid stream. The diglycerides remaining in the vegetable oil enable subsequent production of biodiesel via use of a caustic. In general, production of biodiesel via use of a caustic is much faster than use of an acid and, thus, is generally desirable. The fluid stream used to extract phosphate from the oil could be recycled until the stream saturates with the by-products or until the by-products start affecting the process. In some cases, choline phosphates may be separated from the extraction stream and sold as vitamin supplements.

It is noted that a degumming operation of crude vegetable oil or fat may be performed with either phospholipase C (PLC), a combination of phospholipase A1 (PLA1) and phospholipase A2 (PLA2), or a combination of all three. In particular, PLC will cleave the glycerin phosphorous bond, leaving diglyceride in the oil and putting the alkanolaminephosphate group in the aqueous stream. A combination of PLA1 and PLA2 will remove one of the two fatty acid groups and the resulting monoglyceridealkanolaminephosphate is a viscous third phase at the interface of water and oil. For all of the PLC processes and the combination of PLA1 and PLA2 processes conducted in relation to the development of the methods described herein, there were no gums in the oil detected by the hot water precipitation method. It is noted that phospholipase D (PLD) will remove the alkanolamine from the oil, but will leave the diglyceride phosphate and, thus, PLD would not generally be advantageous to use for degumming operations, but PLD may be advantageous for other processes, such as food production. Furthermore, it is noted that although the disclosure provided herein emphasizes use of phospholipase for degumming operations in a fiber conduit reactor, other enzymes may be considered for such a process, such as but not limited to other hydrolases and lipases.

### Example 1

Enzymatic degumming of crude soybean oil was carried out using Lecitase® Ultra enzyme (i.e., a phospholipase A enzyme provided by Novozymes of Denmark). The reaction was carried out at a flow rate of approximately 2:1 (oil:aqueous enzyme solution) at approximately 60° C. The enzyme solution contained 0.0796 wt. % enzymes in deionized water. The reaction was successfully carried out using a fiber conduit apparatus having stainless steel fibers. The aqueous and oil phases were clear, but there was a rag layer of the modified gum. The crude oil sample was tested for degumming effectiveness by hydrolysis test which showed no gums in oil after the process. During the process there was a slight rise in pressure, presumably due to the viscous rag layer of modified gum. Phospholipase A cleaves one of the fatty acid chains of the gum molecules making a less oily gum derivative which hydrates and separates more easily in the rag layer.

### Example 2

An initial study was carried out using phospholipase C type enzyme using the same fiber conduit apparatus as used in Example 1 and at approximately the same flow rate, temperature and enzyme concentration. The hydrolysis test result showed no gums present in the degummed oil sample. It was a much cleaner reaction than what was conducted in Example 1 (i.e., with a clear interface and no rag layer).

### Example 3

Approximately two milliliters per minute of water containing approximately 0.09% of the enzyme phospholipase C was used as the constrained phase in a ½″×12″ fiber reactor containing about 540,000 eight micron (outer diameter) 316 stainless steel fibers. When filtered crude soybean oil was pumped as the free phase at approximately 4 milliliters per minute at approximately 65° C., the processed oil passed the hydrolysis test for gums. Theoretically, the phospholipids (gums) were converted to oil soluble diacylglyceride and water soluble phosphate derivatives. The crude oil was effectively degummed in less than five minutes with instant separation of the phases. The enzyme solution was recycled three times with similar results.

In addition for use in degumming operations, the use of enzymes in a fiber conduit apparatus may be desirable for the production of biodiesel from degummed vegetable oil (i.e., a transesterification process). For instance, one or more lipases may be introduced into a fiber conduit apparatus as well as degummed vegetable oil for the continuous phase and an alcohol in the constrained phase to produce biodiesel. The lipases which may be considered for use in a fiber conduit apparatus for production of biodiesel include but are not limited to Candida antarctica B, (CALB-L, Novozymes, Denmark), Lipase PS (Pseudomonas cepacia, P, fluorescens), from Novo-Nordisk, Baegsvaerd, Denmark, Lipases from MucorjaVanicus (M) from Amano Enzyme Inc., Nagoya, Japan; Rhizomucor miehei lipase (Palatase M) from Amano. Similar to the enzymatic dephosphorylation of vegetable oils or fats process described above, the transesterification process can be performed in a short amount of time (on the order of a few minutes, depending on the length of the fibers), with no strong acids or bases (and, in some cases, void of any acids and bases), and without the need for subsequent distillation or centrifugation steps. Furthermore, the biodiesel production process may be conducted at a relatively moderate temperature between approximately 40° C. and approximately 70° C., with an optimum range between approximately 50° C. and approximately 60° C. The term “approximately” as used herein refers to variations of up to +/−5% of the stated number.

It will be appreciated to those skilled in the art having the benefit of this disclosure that this invention is believed to provide methods and apparatuses for enzymatic chemical processing in fiber conduit apparatuses. Further modifications and alternative embodiments of various aspects of the invention will be apparent to those skilled in the art in view of this description. For example, although the aforementioned discussions give specific application to degumming vegetable oil and fats and the production of biodiesel from degummed oil, the systems and apparatuses described herein are not so limited and may be used to perform any type of enzymatic chemical process which employs contact between two immiscible fluids. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed:

1. A method of chemical processing, comprising:
introducing a first stream proximate a plurality of fibers positioned within a fiber conduit apparatus, wherein the first stream constitutes a phase substantially constrained to exterior surfaces of the fibers;
introducing a second stream into the fiber conduit apparatus, wherein the second stream constitutes a substantially continuous phase that is in contact with and is substantially immiscible with the first stream along the exterior surfaces of the fibers;
introducing an enzyme which unattached to a substrate into the first stream or the second stream, wherein the first stream, the enzyme and the second stream are introduced into the fiber conduit apparatus such that:
the enzyme interacts with a species from one of the first and second streams; and
compositions of the first and second streams are altered;
receiving the altered first and second streams in one or more collection vessels; and
withdrawing separately the altered first and second streams from the one or more collection vessels.

2. The method of claim 1, wherein the enzyme is selected from a group consisting of oxidoreductases, transferases, hydrolases, lyases, isomerases, and ligases.

3. The method of claim 1, wherein the step of introducing the enzyme into the fiber conduit apparatus comprises adding the enzyme to the first stream or to the second stream prior to introducing the enzyme added stream into the fiber conduit apparatus.

4. The method of claim 1, wherein the step of introducing the enzyme into the fiber conduit apparatus comprises introducing an enzyme solution into the fiber conduit apparatus.

5. The method of claim 1, wherein the first stream, the enzyme and the second stream are introduced into the fiber conduit apparatus such that at least a portion of the species is extracted into the other stream.

6. The method of claim 5, further comprising recycling the other stream through the fiber conduit apparatus for further processing.

7. The method of claim 1, wherein the first stream, the enzyme and the second stream are introduced into the fiber conduit apparatus such that the enzyme and reactive species of the first and second streams interact to form at least one new chemical species.

8. The method of claim 7, wherein the enzyme comprises a lipase, and wherein the new chemical species is biodiesel.

9. The method of claim 1, wherein the first stream comprises water, wherein the enzyme comprises a phospholipase, and wherein the second stream comprises crude or refined vegetable oil and/or fat.

10. The method of claim 1, wherein the first stream is void of an acid having a pH less than 2.0 and is void of a base having pH greater than 13.0.

11. The method of claim 1, wherein the first and second streams move co-currently through the conduit apparatus.

12. The method of claim 1, wherein the first and second streams move counter-currently through the conduit apparatus.

13. The method of claim 1, wherein the first stream comprises an alcohol, an amine, a carboxylic acid, a phenol, an ionic liquid and/or water.

14. An apparatus, comprising:
a conduit comprising at least two fluid inlets and one fluid outlet;
a plurality of fibers positioned longitudinally within the conduit between the two fluid inlets and the fluid outlet, wherein the plurality of fibers have different enzymes immobilized thereon, wherein the plurality of fibers are connected to one of the at least two fluid inlets such that fluid introduced into the one fluid inlet flows along exterior surfaces of the plurality of fibers, and wherein another of the at least two fluid inlets is arranged along the conduit such that fluid introduced into the other fluid inlet flows between the plurality of fibers; and
a collection vessel positioned proximate the fluid outlet.

15. The apparatus of claim 14, wherein the different enzymes are selected from a group consisting of oxidoreductases, transferases, hydrolases, lyases, isomerases, and ligases.

16. The apparatus of claim 14, wherein the different enzymes comprise one or more phospholipases.

17. A method of chemical processing, comprising:
introducing a first stream comprising alcohol proximate a plurality of fibers positioned within a fiber conduit apparatus, wherein the first stream constitutes a phase substantially constrained to surfaces of the fibers;
introducing a lipase into the fiber conduit apparatus;
introducing a second stream containing degummed vegetable oil into the fiber conduit apparatus, wherein the second stream constitutes a substantially continuous phase that is in contact with and is substantially immiscible with the first stream, and wherein the first stream, the lipase and the second stream are introduced into the fiber conduit such that the alcohol, the lipase and phospholipids of the degummed vegetable oil interact to form fatty acid short-chain alkyl esters useful as biodiesel;
receiving the fatty acid short-chain alkyl esters, byproducts of the fatty acid short-chain alkyl esters formation, and residual components of the first and second streams in one or more collection vessels; and
withdrawing separately the fatty acid short-chain alkyl esters from the one or more collection vessels.

18. The method of claim 17, wherein the lipase is a phospholipase.

19. The method of claim 17, wherein the first stream is void of an acid having a pH less than 2.0 and is void of a base having pH greater than 13.0.

20. The method of claim 17, wherein the fibers are hydrophilic and the lipase is immobilized on the fibers.

21. A method of chemical processing, comprising:
introducing a first stream proximate a plurality of fibers positioned within a fiber conduit apparatus, wherein the first stream constitutes a phase substantially constrained to surfaces of the fibers;
introducing an enzyme into the fiber conduit apparatus;
introducing a second stream into the fiber conduit apparatus, wherein the second stream constitutes a substantially continuous phase that is in contact with and is substantially immiscible with the first stream, and wherein the first stream, the enzyme and the second stream are introduced into the fiber conduit apparatus such that the enzyme and species of the first and second streams interact to form a chiral product with high enantiometric purity;
receiving the chiral product, byproducts of the chiral product formation, and residual components of the first and second streams in one or more collection vessels; and
withdrawing separately the chiral product from the one or more collection vessels.

22. The method of claim 21, wherein the enzyme is selected from a group consisting of oxidoreductases, transferases, hydrolases, lyases, isomerases, and ligases.

23. A method of chemical processing, comprising:
introducing an aqueous first stream comprising an enzyme proximate a plurality of fibers positioned within a fiber conduit apparatus, wherein the aqueous first stream is void of any acids, bases, and solvents, and wherein the aqueous first stream constitutes a phase substantially constrained to surfaces of the fibers;
introducing a second stream of vegetable oil or fat into the fiber conduit apparatus, wherein the second stream constitutes a substantially continuous phase that is in contact with and is substantially immiscible with the first stream, and wherein the first stream and the second stream are introduced into the fiber conduit apparatus such that:
the enzyme converts phospholipids of the second stream into diglycerides and phosphtides; and
at least some of the phosphatides transfer into the first stream;
receiving the altered first and second streams in one or more collection vessels; and
withdrawing separately the altered first and second streams from the collection vessels.

24. The method of claim 23, wherein the enzyme is selected from a group consisting of hydrolases, lipases, and phospholipases.

25. The method of claim 24, wherein the enzyme is phospholipase C.

26. The method of claim 24, wherein the enzyme is phospholipase A1, and wherein the aqueous first stream further comprises phospholipase A2.

27. The method of claim 26, wherein the aqueous first stream further comprises phospholipase C.

28. The method of claim 23, wherein the second stream comprises crude vegetable oil and/or crude fat.
